(19) United States
(12) Patent Application Publication
(10) Pub. No.: US 2010/0130984 A1
(43) Pub. Date: May 27, 2010

(54) SYSTEMS AND METHODS FOR WIRELESS OPHTHALMIC SURGICAL MODULES

(76) Inventors: John Alan Ritter, Des Peres, MO (US); Thomas G. Moore, JR., Kirkwood, MO (US)

Correspondence Address: Michael L. Smith;Bausch & Lomb Incorporated, One Bausch & Lomb Place, Rochester, NY, 14604-2701, US

(21) Appl. No.: 12/275,868

(22) Filed: Nov. 21, 2008

(51) Int. Cl. A61F 9/007

(52) U.S. Cl. 606/107; 604/22; 606/1

(57) ABSTRACT

An ophthalmic surgery system for wirelessly supplying power to at least one surgical module during ophthalmic surgery. The ophthalmic surgery system includes a power module having a power transmitter configured to generate an electromagnetic field in at least a space proximal to the power module, a surgical module including a power receiver, and a surgical console in communication with the surgical module. Power is wirelessly induced in the power receiver when the surgical module is within the space, such that at least one component included in the surgical module is substantially powered by the induced power.

### FIELD

The present disclosure is directed to systems and methods for wirelessly providing power to ophthalmic surgical modules.

### DESCRIPTION OF RELATED ART

The statements in this section merely provide background information related to the present disclosure and may not constitute prior art.

For an ophthalmic surgery system effective for multiple ophthalmic surgery techniques, several components are often included in the ophthalmic surgery system and usable in one or more of the ophthalmic surgery techniques. Each component usually includes power cables and/or physical contacts for supplying power to the component. Components may be pluggable to make removal and/or maintenance of the component more efficient. In most instances, the cables and/or physical contacts associated with the components are connected to the ophthalmic surgery system. The component may be connected, disconnected, and/or reconnected several times per day, daily, or periodically over a lifetime of the ophthalmic surgery system, depending on the particular function of the component and the type of ophthalmic surgery techniques to be performed. In general, as the number of components included in the ophthalmic surgery system increases, the number of cables and/or physical contacts included in the ophthalmic surgery system also increases.

Elimination of cables and/or physical contacts between the surgical components and the ophthalmic surgery system during procedures has been accomplished by including various types and sizes of batteries in the various surgical components. The ophthalmic surgery system generally includes a docking station having one or more cables and/or physical contacts, such that a surgical component may be periodically returned to the docking station to recharge the batteries included therein. Periodic charging may be required daily or more/less often depending on the characteristics of the batteries and the type of ophthalmic procedure to be performed.

In order to take advantage of the modularity of various surgical components, it is desirable to reduce a number of power cables associated with the various surgical components

### BRIEF DESCRIPTION OF THE DRAWINGS

The drawings described herein are for illustration purposes only and are not intended to limit the scope of the present disclosure in any way.

FIG. 1 is a block diagram of an exemplary embodiment of an ophthalmic surgery system according to the present disclosure; and

FIG. 2 is a block diagram of an exemplary embodiment of a method of wirelessly supplying power to a surgical module according to the present disclosure.

### DETAILED DESCRIPTION OF THE PREFERRED EMBODIMENT

The following description is merely exemplary in nature and is not intended to limit the present disclosure, application, or uses.

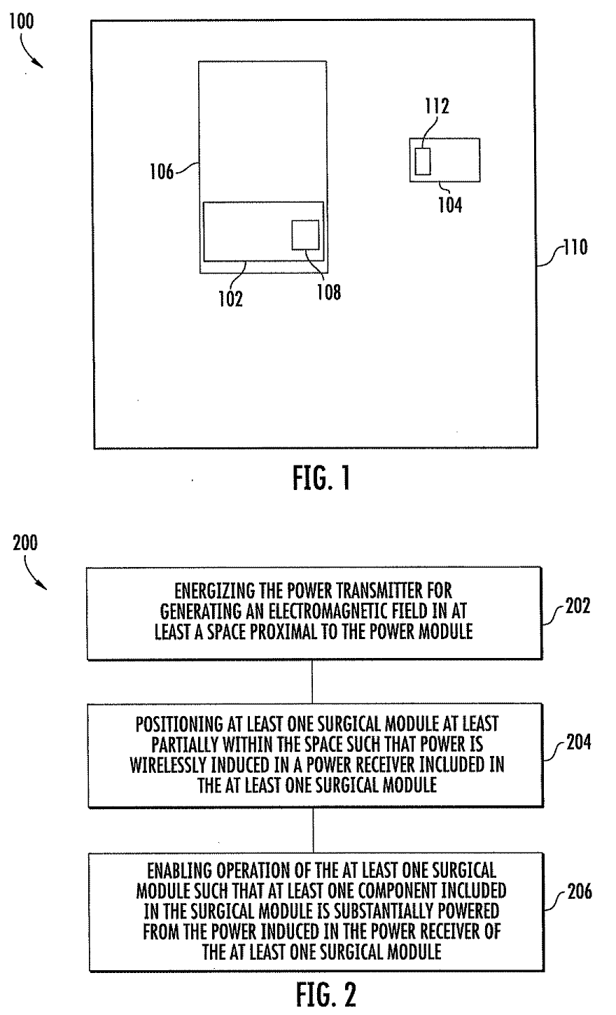

According to one exemplary embodiment of the present disclosure, an ophthalmic surgery system for wirelessly supplying power to at least one surgical module during ophthalmic surgery is illustrated in FIG. 1, and referenced 100. The ophthalmic surgery system 100 includes a power module 102, a surgical module 104, and a surgery console 106 in communication with the surgical module 104. The power module 102, includes a power transmitter 108 configured to generate an electromagnetic field in at least a space 110 proximal to the power module 102. The surgical module 104 includes a power receiver 112. When the surgical module 104 is within the space 110, power is wirelessly induced in the power receiver 112, such that one or more components included in the surgical module 104 are substantially powered by the induced power. By providing power to the surgical module 104 in this manner, power cables and/or physical contacts between the power module 102 and the surgical module 104 are eliminated. Such wireless power supplies are known, such as WiTricity developed by the Massachusetts Institute of Technology.

In various embodiments of the present disclosure, a surgical module may include a scissor module, a light module, a phacoemulsification module, a pump module, a surgical handpiece, a foot-pedal, or a different modular piece of equipment utilized during one or more ophthalmic surgery techniques. While the ophthalmic surgery system 100 includes only one surgical module, it should be appreciated that a different number of surgical modules may be employed in other embodiments of the present disclosure. In some embodiments, a number of surgical modules may be based on an amount of current/voltage supplied to a power transmitter and/or characteristics particular to the power transmitter.

Referring again to FIG. 1, the power transmitter 108 includes a coil, and the power receiver 112 includes a coil. The coil included in the power transmitter 108 has substantially the same resonant frequency as the coil in the power receiver 112. In this manner, transfer of power between the two coils may be more efficient than power transfer between two coils with substantially different resonant frequencies. It should be appreciated that various types of coils with matching and un-matching resonant frequencies may be employed in an ophthalmic surgery system according to other embodiments of the present disclosure.

The electromagnetic field is provided in the space 110, represented by a rectangular block in FIG. 1. It should be appreciated that the size and shape of an electromagnetic field is dependent on at least the type and configuration of a power transmitter and voltage/current supplied to the power transmitter. The size and shape of an electromagnetic field may also be defined by boundaries or otherwise limited to prevent and/or inhibit an electromagnetic field from interacting with other electronic equipment and/or persons within a range of a power transmitter.

While the power module 102 is illustrated within the surgery console 106, it should be appreciated that in other embodiments of the present disclosure, a power module may be disposed partially within the surgery console or separate from the surgery console.

The surgery console 106 is in communication with the surgical module 104 to allow commands to be relayed to and/or from the surgical module 104. One or more commands may be provided to enable use of the surgical module 104. For example, during a phacoemulsification procedure, a command may be relayed from a foot-pedal through the surgery console to a surgical handpiece for vibrating a phacoemulsification needle included in the surgical handpiece. In the same example, the surgical handpiece may also provide feedback information to the surgery console. In another example, a foot-pedal included in a surgical module may provide single-direction communication to a surgery console. It should be understood that communication may be single-directional or bidirectional between a surgical module and a surgery console and/or between multiple surgical modules depending on the particular type and implementation of the one or more surgical modules.

Referring again to FIG. 1, communication between the surgery console 106 and surgical module 104 is completed through a wireless network. Accordingly, communication cables, in addition to the power cables, may be eliminated from between the surgical module 104 and the surgery console 106, thereby providing a completely unwired surgical module in some instances. In one exemplary implementation, a surgical handpiece for retinal surgery may include wireless power from a surgery console and wireless communication from the surgery console. In this manner, for the duration of the retinal surgery, the surgeon is able to maneuver the surgical handpiece without being restricted by any cables coupling the surgery console. In another example, for phacoemulsification surgery, power and communication cables can be eliminated leaving only aspiration and irrigation tubing to the surgical handpiece.

The wireless network may include Bluetooth, ZigBee, or a different type and size personal wireless network. A separate network may be provided for each surgical module and/or each type of surgical module included in an ophthalmic surgery system. Alternatively, the ophthalmic surgery system 100 includes a single wireless network to communicate with the surgical module 104 and any other surgical modules added thereto. In some embodiments, wired communication may be included in an ophthalmic surgery system based on one or more operating requirements of a particular ophthalmic surgery technique.

According to one embodiment of the present disclosure, a method of wirelessly supplying power from a power module to at least one surgical module during ophthalmic surgery is illustrated in FIG. 2. Method 200 includes step 202 for energizing a power transmitter for generating an electromagnetic field in at least a space proximal to the power module, step 204 for positioning at least one surgical module at least partially within the space, such that power is wirelessly induced in a power receiver included in the at least one surgical module, and step 206 for enabling operation of the at least one surgical module, such that at least one component included in the surgical module is substantially powered from the power induced in the power receiver of the at least one surgical module.

According to method 200, each and every component included in the surgical module is powered solely from the induced power. In other embodiments, fewer components included in a surgical module may be powered by the induced power depending on the availability of alternate power and/or a power scheme or sequence for components included in a surgical module that would make utilizing the induced power impractical or inefficient.

By implementing any or all of the teachings described above, a number of benefits and advantages can be attained, including improved reliability, reduced down time, elimination or reduction of redundant components or systems, avoiding unnecessary or premature replacement of components or systems, and a reduction in overall system and operating costs.

1. An ophthalmic surgery system for wirelessly supplying power to at least one surgical module during ophthalmic surgery, the ophthalmic surgery system comprising:
a power module having a power transmitter configured to generate an electromagnetic field in at least a space proximal to the power module;
a surgical module including a power receiver;
a surgical console in communication with the surgical module; and
power being wirelessly induced in the power receiver when the surgical module is within the space, such that at least one component included in the surgical module is substantially powered by the induced power.

2. The invention of claim 1, wherein each of the power transmitter and the power receiver include a coil having a resonant frequency, said coils having substantially the same resonant frequencies.

3. The invention of claim 1, wherein the power module is disposed at least partially within the surgery console.

4. The invention of claim 3, wherein the surgical console is configured to communicate with the surgical module through a wireless network.

5. The invention of claim 4, wherein the surgical module includes one of a surgical handpiece and a foot-pedal.

6. The invention of claim 3, wherein the surgical module includes at least one of a scissor module, a light module, a pump module, and a phacoemulsification module.

7. The invention of claim 1, further comprising a second surgical module including a second power receiver, wherein power is wirelessly induced in the second power receiver when the second surgical module is within the space, such that at least one component included in the second surgical module is substantially powered by the induced power from the second power receiver.

8. A method of wirelessly supplying power from a power module having a power transmitter to at least one surgical module during ophthalmic surgery, the method comprising:
energizing the power transmitter for generating an electromagnetic field in at least a space proximal to the power module;
positioning at least one surgical module at least partially within the space such that power is wirelessly induced in a power receiver included in the at least one surgical module; and
enabling operation of the at least one surgical module such that at least one component included in the surgical module is substantially powered from the power induced in the power receiver of the at least one surgical module.

9. The invention of claim 8, wherein each of the power transmitter and the power receiver include a coil having a resonant frequency, said coils having substantially the same resonant frequencies.

10. The invention of claim 8, wherein enabling operation of the at least one surgical module includes wirelessly communicating with the surgical module through a surgery console.

11. The invention of claim 10, wherein the power module is disposed at least partially within the surgery console.

12. The invention of claim 8, wherein the surgical module includes one of a surgical handpiece, a foot-pedal, a scissor module, a light module, a pump module, and a phacoemulsification module.
